(19) United States
(12) Patent Application Publication
(10) Pub. No.: US 2007/0295620 A1
(43) Pub. Date: Dec. 27, 2007

(54) Medical Package, a Medical Container, a Medical Package System and Methods for Removal of a Medical Component from a Container

(75) Inventors: Richi Collet, Venray (NL); Niklas Hofverberg, Molndal (SE); Kent Engstrom, Molndal (SE); Bjorn Bengtzelius, Molndal (SE)

Correspondence Address: BIRCH STEWART KOLASCH & BIRCH, PO BOX 747, FALLS CHURCH, VA, 22040-0747, US

(73) Assignee: Astra tech AB, Molndal (SE)

(21) Appl. No.: 11/662,727

(22) PCT Filed: Jul. 7, 2005
(86) PCT No.: PCT/SE05/01123

§ 371 Date: May 11, 2007

(30) Foreign Application Priority Data
Sep. 15, 2004 (SE) 0402230-7

(51) Int. Cl. A61B 19/02

(52) U.S. Cl. 206/63.5; 206/368

(57) ABSTRACT

According to the present application, a medical package including a container (1) and a medical component (6), in particular a dental implant component, is disclosed. Said container (1) in a closed position thereof forms a component compartment (2) for said medical component (6) and an access channel (5) for an external instrument. Said access channel (5) is dimensioned to prevent said component (6) from passing through the access channel (5) and to allow said instrument to be inserted through the channel (5) and brought into engagement with the component (6) while the container (1) is in its closed position. Said container (1) is transferable to an open position while maintaining said engagement of the external instrument with the component (6), and said component (6), in the open position of said container (1), is removable from the container (1) by said external instrument. Furthermore, a medical package system including an external instrument, a container (1) and methods for removing a medical component (6) from a container (1) are disclosed.

### FIELD OF THE INVENTION

A medical package including a container and a medical component, in particular a dental implant component, is disclosed. Said container in a closed position thereof forms a component compartment for said medical component. Furthermore, a medical package system is disclosed that includes a medical package and an external instrument used for removing a medical component from said package. Finally, a container for a medical component, and methods for removal of a medical component from such containers are disclosed.

### BACKGROUND OF THE INVENTION

Packages and containers of the above-mentioned design are used for transportation and storing of different types of products. When it comes to medical components, they are sometimes to be kept sterile. There are particular demands regarding the sealing of the package containing such a medical component, as well as the handling of said medical component when extracted from the package. It is common to use a double package system, wherein a sterile component is held in an inner package. The inner package is in turn surrounded by an outer package. The outer package is to be kept accurately sealed, so that it may be transported in a non-sterile environment without impairing the sterile environment inside the outer package. Thus the inner package will be kept sterile, and may, after opening of the outer package, be taken into a sterile environment such as a surgery room. Finally, the sterile component may be extracted from the inner package. The various aspects of the present inventive concept are especially, not exclusively intended for use as an inner package in a double package system as described above.

U.S. Pat. No. 6,012,580 (Peters) discloses a universal dispenser for an implant or other item, which includes two clamshell halves pivoted together at a hinge. The halves form an elongated opening for accommodating an implant or other item when the package is closed. The size of the opening is slightly smaller than the item or implant to be stored therein. In a closed position, projections on one half are frictionally retained by recesses in the other half, to keep the clamshell halves in the closed position. For extracting the implant from the package, the side edges of the clamshell halves are squeezed together, whereby the size of the opening is expanded, and the implant may fall out of said opening. This package is initially closed by folding about a hinge located on one side of the package. Opening of the package, for extracting the product, on the other hand is done by squeezing the side edges towards each other parallel to the axis of the hinge for closing the package.

U.S. Pat. No. 5,405,005 (White) discloses a package for a hip prosthesis. A part of the prosthesis extends out of the package, so that distal sleeves can be added onto the implant while still resting in the package. The package has two trays and a hinged web component, which snaps around the implant to encase it. Each tray has a recess that conforms generally to the outer surface of the prosthesis.

In U.S. Pat. No. 4,872,551 (Theros), a clamshell blister package is disclosed that is partly surrounding a pliers or similar tool. As in US 5 405 005, the tool handles project out through the blister package for easy access. The part of the tool that is in need of protection from the surrounding environment is kept within a pair of opposing mirror-image panels that are joined along a base portion having recesses for said handles.

A disadvantage with the teachings in the abovementioned U.S. Pat. No. 6,012,580 is that it is difficult to handle the component in a controlled manner while extracting it from the package. This kind of package requires an intermediate storage, such as a tray, which the component can fall onto and later be picked up from for final installation. As a general remark, prior art packages may require an extra personnel for preparation or assisting during the surgery.

Further, in particular when it comes to relatively small components, such as dental components, these are usually not installed in the patient directly by the hand of a surgeon. Instead, an instrument of some kind, for example a screwdriver or a holder, is coupled to the component prior to installation. This procedure may sometimes be troublesome and requires precision handling of the user. This is particularly so when the component is lying freely on said tray and rolls around when it is to be captured, or be picked up, by said holder.

Also, when the component to be stored in the container is small, the size of the container itself is usually also small in order to save space. However the container must not be too small, since the surgeon's finger technique sets a bottom limit to the container size. Containers like the ones disclosed in the above-mentioned U.S. Pat. No. 5,405,005 and U.S. Pat. No. 4,872,551 patents are likely suitable for their purpose when such rather large sized components are placed therein. However, if they were to be used for smaller sized components, also the containers are most likely reduced in size. However, they are probably not suited for such a size reduction since the opening means also becomes correspondingly size-reduced, and are hence likely not suitable for such small type of components.

### SUMMARY OF THE INVENTION

Thus, there is a need for a package for medical components, especially relatively small sized components that are easy to handle during surgery and surgery preparations. Furthermore, a package that is admitting control over the medical component during the opening of the package would be advantageous.

The object of the present inventive concept is to provide a package for medical components that constitutes an improvement in one or several of the above-mentioned respects.

According to one aspect of the inventive concept, a medical package including a container and a medical component, in particular a dental implant component, is disclosed, wherein said container in a closed position thereof forms a component compartment for said medical component and an access channel for an external instrument, said access channel being dimensioned to prevent said component from passing through the access channel and to allow said instrument to be inserted through the channel and brought into engagement with the component while the container is in its closed position; and

wherein said container is transferable to an open position while maintaining said engagement of the external instrument with the component;

whereby said component, in the open position of said container, is removable from the container by said external instrument.

Such a package for medical components is well suited for the handling of components that are of a smaller size, i.e. components such as dental implant components or the like. This way the component can be engaged with the external instrument even before the container is opened, and thus the user of the package may efficiently obtain handling control over the component at an early stage. This is an advantage in relation to for example the container disclosed in U.S. Pat. No. 6,012,580, since no intermediate storage such as a tray is needed in this aspect of the present inventive concept, onto which said component must fall when extracting it from the container when using the latter.

The expression “engagement” is in this respect and throughout this application to be understood to include a component-carrying function sufficient to effect the removal of the component by the instrument after opening of the container.

As stated above, the access channel is dimensioned to prevent said component from passing through the access channel while the container is in its closed position. Thus, the component cannot unintentionally fall out when the container is closed. However, the access channel is dimensioned to allow the external instrument to be inserted through the channel and brought into engagement with the component while the container still is in its closed position.

After establishing the engagement between the instrument and the component within the closed container, the container may be opened to remove the component. This may be done while the external instrument is kept in its engagement with the component. Thus, the control over the component is not lost. When the container has been transferred to its open position, the external instrument may be used to remove the component from the open container, for example to lift it up from the component compartment. Thereafter, the instrument carrying the component may be used for bringing the component to an intended site.

Particularly if the component is a medical component that may need to be kept in a sterile environment, the risk of dropping and thus loosing the component when removing it from a container, such as when dropping the component in U.S. Pat. No. 6,012,580 onto a tray or when trying to get it into engagement with an external instrument on said tray either, is essentially eliminated.

When a package according to this inventive concept C has to be kept sterile all through the process of transportation, the package may be kept sterile through the use of an outer container, even though the access channel may form a channel to the surrounding environment inside the outer container. However said channel leads through the container only, not through the outer container. The component and the inside of the package may be kept sterile by sealing the openings and joints by adding a sterility ensuring sealing closure, such as an adhesive tape or a foam. The closure is later broken when opening the package. If such a sterile environment is not a necessity, the package might be used as it is.

It may be, in the use of said package, that the hand of the user may not come in contact with the component. Only the external instrument, that may be pre-sterilized if necessary, may come in contact with the component. Cleanliness is thus an advantage that may be obtained when using this package.

Furthermore, if the external instrument is possible to handle and open by one hand only, and the container is possible to also be held by one hand only, the package may advantageously be handled by one person only, performing both the package opening and the component removal.

It may be advantageous if the component compartment is structured and adapted to hold said medical component in an essentially fixed position within the component compartment, when the container is in its closed position. This may protect the component from tumbling around in said compartment and hence protect it from damage.

If said medical component presents an engagement portion and said instrument presents an engagement portion, which engagements portions are structured and adapted to be brought into engagement with each other, a simple engagement mechanism may be achieved for the container.

It may be valuable if the component compartment is structured and adapted to retain said medical component to such an extent that, in the closed position of the container, the engagement portion of the medical component is kept in an access position facing the access channel to be engaged by the instrument. In this position of the component in relation to the container, an easy access of the component by the instrument may be achieved. There is hence no need for orientating the component first within the compartment in relation to the access channel when engaging the component by the external instrument. This simplifies the handling of the package.

An advantage may be gained if said engagement portion of the medical component and said engagement portion of the instrument are structured and adapted to provide a rotationally fixed engagement with respect to an axis essentially parallel to the access channel. This way it is possible to use the external instrument for the removal of the component from the container also in situations in which the user needs control over the component by the use of the same instrument also when the component has been removed from the container.

An advantage may be gained if said medical component is structured and adapted to be screwed into a prepared site and said engagement is a rotationally fixed engagement allowing said medical component, when removed and still engaged by the instrument, to be screwed at least partly into the prepared site by the use of the same instrument. This way it is possible to use the same external instrument for the removal of the component from the container also in a situation in which the component is adapted to be screwed into place after said removal from the container, such as when screwed into, for instance, a fixture or the fixture itself is screwed directly in the bone tissue. One instrument less is hence required for the handling of the component.

It may be convenient if said container is structured and adapted to be opened by one hand only, preferably by two fingers of one hand. This would further emphasize the simplicity of the handling of the container. Especially, embodiments may be structured such that the container may be held and opened by one hand, while the external instrument is held by the other hand for engaging and subsequently removing the component from the container.

Conveniently, said container may be structured and adapted to be opened by means of application of a pressure against opposite sides of the container. When pressure is used rather than for instance tension forces for opening the container, the hand of the user is normally stronger in that respect, and thus the container is better suited for easy handling.

In order to protect the component within the compartment it is beneficial for said container to be structured and adapted to be opened by means of the application of a pressure against opposite sides of a maneuver section of the container, said maneuver section of the container being spaced from the component compartment of the container, whereby said pressure is not applied on the compartment when the container is opened.

The opening maneuver of the package may be simplified if said container comprises first and second container parts being pivotable relatively to each other about a pivot axis for bringing the container to its open position, by the application of a pressure on a maneuver section of the container, said maneuver section and said component compartment being located on opposite sides of said pivot axis. This way a lever is achieved by the maneuver section in relation to the pivot axis. The longer the lever, the lesser the force needed in order to open the container and uncover the component compartment. The parts of the container on the opposite side of the pivot axis in relation to the maneuver section may also work as a lever and thus the uncovering of the compartment is easy accomplished. It is contemplated that the pivot axis may not necessarily be a fixed point in the geometrical space in relation to the package, but surfaces, such as ridges, that ride on one another, thus accomplishing a similar lever effect. However, the “pivot axis” in this latter case is somewhat displaced in space during the opening maneuver. It should furthermore be noticed that the “pivot axis” need not be a rigid point, but also a somewhat flexible part of the container that may be slightly deformed during the opening maneuver.

The length of the lever that is created by the maneuver section may be adapted to the size of the user's hand in such a way that if, for instance, the thumb and the index finger of one hand are used in the opening procedure of the container, the fingertips are somewhat larger than the maneuver section. The user may thus apply some pressure on both sides of the pivot axis and further improve the control over the opening procedure, i.e. the pressure applied to the container on the opposite side of the pivot axis in relation to the maneuver section works as an anvil.

If only the thumb and the index finger of one hand of the user are used in the opening procedure of the container, and the maneuver section is somewhat smaller than the fingertips thus controlling the grip around the package, it is advantageous that the user may hold the package with the one hand, and need not change the grip around the package when he or she wants to open it. The user may slightly adjust the grip enough to displace the force onto the maneuver section and open the package.

Said first container part may be located essentially entirely on one side of a plane coinciding with said pivot axis and wherein said second container part is located essentially entirely on an opposite side of said plane.

It may be beneficial that an interface between said first and second container parts together define a geometrical medial plane of the closed container;

said pivot axis is located essentially in said medial plane;

said medial plane and said pivot axis together define a geometrical lateral plane coinciding with the pivot axis and being transverse to said medial plane;

said first and second container parts each present a main portion being located essentially on a first side and a second side, respectively, of said medial plane;

said main portions are located on a first side of said lateral plane facing away from the maneuver section of the container,

said main portions form said component compartment and said channel of the container;

said first and second container parts each present also a maneuver portion located on an opposite second side of said lateral plane, said maneuver portions together forming said maneuver section of the container and being adapted to be rotated against each other upon application of said pressure.

The component compartment may located between the access channel and said pivot axis. Thus, no parts may obstruct the access of the compartment through the access channel. Furthermore, said access channel and said pivot axis may be located essentially in a common geometrical plane.

If said pivot axis is located in a geometrical medial plane formed by an interface of said first and said second portions of the container in the closed position thereof, the container is opened by rotation about said pivot axis by equal displacements on each side of the axis and plane. In this case, the component is uncovered on both sides thereof and may easily be removed from the container.

In order to guide the external instrument in relation to the component when introducing the former into the access channel towards the component, it may be advantageous that said access channel is tapering towards the component compartment. To get the most guidance for the instrument, the access channel may present a smallest cross section situated adjacent to the component compartment. A way of achieving guidance of said external instrument may be to allow at least a portion of the access channel to be designed so as to correspond to an outer contour of the instrument, whereby said channel provides a guide for positioning said instrument in relation to said component.

In order to provide for easy production of the container and also for easy assemblage of the package, i.e. including the component, said container may be formed as a one-piece unit. One way of accomplishing a foldable one-piece container may be to manufacture the container of a pliable or flexible material, such as a plastic. The container may be designed to be flexible, such as by giving it a design that is resilient.

The container of the package may advantageously comprise:

a first part and a second part forming together said component compartment, and

an intermediary part connecting said first and second portions.

Easy handling of the container may be achieved if said intermediary portion is hingedly connected to the first and second container part. In this way the container is given inherent hinges, about which the container's first and second parts may be rotated in order to close the container. The hinges need not coincide with a possible pivot axis for the transferal of the container from its closed position to its open position for removal of the component. However, if the pivot axis is designed not to coincide with the hinges, the transferal of the container from its closed position to its open position is not performed in a similar manner as the transferal from its initial open position to its closed position. Furthermore, it is contemplated that the hinges may be designed so that the initially empty container may lie flat open on a surface, which in turn may simplify the adding of the component into the container. In this case, the hinges will be positioned essentially on the outer surface of the subsequently closed container.

For easy production and handling, said first, second and intermediary parts may be formed as a one-piece unit.

In order to accomplish that a transferal of the container from its closed position to its open position is performed in a for the user predictable manner, said intermediary container part forms a resilient support means for obtaining movement stabilization when the container is brought from its closed position to its open position. The same intermediary container part may be advantageous also when transferring the container from its initial open position to its closed position, i.e. when it is folded, since a predictable behavior of the container is also needed at this stage. For instance, it may be useful to be able to control the relative positions of the first and second container parts in order for the component not to fall out of the component compartment.

The package may comprise locking means for preventing unintentional opening of the container. The locking force of the locking means may be adjusted to correspond to a suitable force that feels “acceptable” to the user. If said locking means are formed in one piece with the container, the production of the package is facilitated. To even further simplify the production and to use common and thoroughly tested means to lock the package, said locking means may comprise at least one of frictional locking means and snap lock means. Since the instrument has control over the component within the component compartment by the engagement portion, the locking force that need to be overcome during the opening procedure may be increased in order to limit the risk for unintentional opening of the container.

In order to simplify the use of the package and the possibility to see what type of component that is contained therein, the container may, at least partly, be made of a transparent material. The container may furthermore be a blister-type container.

Since the package is especially suitable to be used in connection with small-sized components, due to its simple design, said medical component may be well suited to be a component of a dental implant system that often has a size in the range of a couple of millimeters. Such small sized components may be abutments, healing caps or cover screws.

In order to keep sterile conditions around the component, no part of said component, in the closed position of said container, protrudes outside of said container. Another advantage may be that the component, whether or not constrained to sterile conditions, is better protected from outer influence such as a shock.

According to a second aspect of the inventive concept, a medical container for containing a medical component, in particular a dental implant component, wherein said container in an initial open position thereof is adapted to receive said component, said container in a closed position thereof forms a component compartment for said medical component, and said container, in a component extraction position thereof, is adapted to present said component compartment for component extraction there-from,

wherein said container is transferable from said initial open position to said closed position in one geometrical plane, and is transferable from said closed position to said component extraction position in essentially the same geometrical plane.

Such a package for medical components is well suited for the handling of components that are of a smaller size, i.e. components such as dental implant components or the like. Furthermore, the container is admitting control over the medical component during the opening thereof. The user may know in what direction the container is opening and may thus not be taken by surprise.

The object of the present inventive concept is provided by the medical container according to the second aspect of the inventive concept although this aspect does not comprise an access channel in the container.

Where applicable, the features, structures or advantages that are described in relation to the first aspect of the inventive concept may also be embodied in the second aspect of the inventive concept.

According to a third aspect of the inventive concept, a medical container for containing a medical component, in particular a dental implant component, wherein said container in an initial open position thereof is adapted to receive said component, said container in a closed position thereof forms a component compartment for said medical component, and said container, in a component extraction position thereof, is adapted to present said component compartment for component extraction there-from,

wherein said container is transferable from said initial open position to said closed position about hinge means, and is transferable from said closed position to said component extraction open position about a pivot axis, wherein the extension of said hinge means and said pivot axis are essentially parallel to and separated from one another.

Such a package for medical components is well suited for the handling of components that are of a smaller size, i.e. components such as dental implant components or the like. Furthermore, the container is admitting control over the medical component during the opening thereof. The user may know in what direction the container is opening and may thus not be taken by surprise.

The object of the present inventive concept is provided by the medical container according to the third aspect of the inventive concept although this aspect does not comprise an access channel in the container.

Where applicable, the features, structures or advantages that are described in relation to the first aspect of the inventive concept may also be embodied in the third aspect of the inventive concept.

According to a fourth aspect of the inventive concept, a medical container for containing a medical component, in particular a dental implant component, wherein said container comprises an maneuver section, a first body, and a second body, said first and second body being pivotable relatively to each other about a pivot axis between a closed position and an open position in which the component is extractable from the package, wherein in the closed position,

the first and second body form a component compartment for containing said component, and

wherein the pivot axis is provided between the maneuver section and the component compartment, such that an opening force applied to said maneuver section is transferred via the pivot axis for bringing the first and second body from the closed position to the open position.

Such a package for medical components is well suited for the handling of components that are of a smaller size, i.e. components such as dental implant components or the like. Furthermore, the container is admitting control over the medical component during the opening thereof. The maneuver section may work as a lever in relation to the pivot axis, and when applying a pressure to the maneuver section the user may adjust the applied force both in strength and in position to the pivot axis and thus control the opening procedure. When in control over the opening, the user may limit the risk for dropping or otherwise loosing any component contained within the container.

The object of the present inventive concept is provided by the medical container according to the fourth aspect of the inventive concept although this aspect does not comprise an access channel in the container.

Where applicable, the features, structures or advantages that are described in relation to the first aspect of the inventive concept may also be embodied in the fourth aspect of the inventive concept.

According to a fifth aspect of the inventive concept, a medical package system is disclosed, comprising:

a package including a container and a medical component, in particular a dental implant component, and an instrument for removing said component from the container,

wherein said container in a closed position thereof forms a component compartment for said medical component and an access channel for said instrument, said access channel being dimensioned to prevent said component from passing through the access channel and to allow said instrument to be inserted through the channel and brought into engagement with the component while the container is in its closed position; and

wherein said container is transferable to an open position while maintaining said engagement of the instrument with the component;

whereby said component, in the open position of said container, is removable from the container by said instrument.

Such a medical package system is well suited for the handling of medical components that are of a smaller size, i.e. components such as dental implant components or the like. The medical package, including a container and a medical component, involves the same advantages as has been discussed above in relation to the first aspect of the present inventive concept, such as the minimizing of the risk of dropping or loosing the component while removing it from the container. The medical package system, however, also comprises an instrument for removing said component from the container. This instrument is suitable to use when removing the component from the container by first accessing the component through the access channel, and engaging the component before opening the container. The user may also limit the touching of the component by other means than the instrument, which if necessary, may be pre-sterilized. This further emphasizes the advantage of cleanliness that is gained in relation to the package system.

The object of the present inventive concept is provided by the package system according to the fifth aspect of the inventive concept although this aspect does not comprise an access channel in the container.

Where applicable, the features, structures or advantages that are described in relation to the first aspect of the inventive concept may also be embodied in the fifth aspect of the inventive concept.

According to a sixth aspect of the present inventive concept, a method for removing a medical component, in particular a dental implant component, from a container is disclosed, comprising:

in a closed position of said container, inserting an instrument through an access channel of said container for bringing said instrument into engagement with the component;

bringing said container to an open position while maintaining said engagement; and

removing the component from the container by said instrument.

The present inventive method provides a simple manner of accessing and removing a medical component from a container, which method at the same time secures the control over the component already before the container is opened. The control over the component at an early stage of the opening procedure of the container, secures that the component is not lost or unintentionally dropped onto an environment for which it is not prepared. It may even break on impact. If the component would be pre-sterilized and in need of being kept sterilized until it has reached its target destination, dropping it onto a non-controlled surface would normally mean loosing the component. This could in turn mean that a new component must be ordered, and for instance a surgical operation may need to be made all over again at a later stage, thus causing unnecessary pain and agony for the patient.

The control over the component is ensured by accessing the component through an access channel in the container and engaging the component by an instrument. This may be done when the container is still closed. After this stage, when the container is brought into an open position, the component is still being engaged by the instrument all trough the opening process, and the component may be removed by the use of the same instrument after the opening process has been finished.

Where applicable, the features, structures or advantages that are described in relation to the first aspect of the inventive concept may also be embodied in the sixth aspect of the inventive concept.

It may be advantageous if the inventive method would comprise:

initially holding the closed container with one hand and the instrument with the other hand; and

performing the opening of the container by the use of said one hand only.

Since this means that only two hands are needed in order to perform the operation, one person can by him- or herself perform said operation.

According to a seventh aspect of the present inventive concept, a method for removing a medical component, in particular a dental implant component, from a container is disclosed, said container comprising first and second container parts which form a compartment for said component and which are pivotable relatively to each other about a pivot axis for bringing the container to its open position, comprising:

bringing the container from its closed position to its open position by the application of a pressure on a maneuver section of the container, wherein said maneuver section and said component compartment being located on opposite sides of the pivot axis; and

removing the component from the opened container.

According to the present method, a method for opening a container for a medical component is disclosed, in which the user of the container may bring the container to its open position by only applying a pressure on a maneuver section on the container. By applying pressure on the maneuver section, instead of for instance tension forces on two parts of said section, a simple mode of operation is used that is not in need of a good finger technique.

Where applicable, the features, structures or advantages that are described in relation to the first aspect of the inventive concept may also be embodied in the seventh aspect of the inventive concept.

It may be advantageous if the method comprises:

in the closed position of the container, inserting an instrument through an access channel of said container for bringing said instrument into engagement with the component;

bringing said container to its open position by the application of said pressure on the maneuver section while maintaining said engagement; and

removing the component from the container by said instrument.

An access channel that allows access of the component by an instrument already when the container is closed, gives the user of the container control over the component before the container is opened. Thus minimizing the risk for dropping or otherwise loosing the component during the opening procedure of the container.

### BRIEF DESCRIPTION OF THE DRAWINGS

By way of example, embodiments will now be described with reference to the accompanying drawings in which:

FIG. 1 is an isometric view of a package according to a first exemplary embodiment with a cover screw in the component compartment.

FIG. 2 is an isometric view of the package in FIG. 1 in an initial open position with a cover screw in the component compartment.

FIGS. 3a-3d are schematic side views of a container according to the present inventive concept in different opening or closing positions.

FIGS. 4a-4d are isometric views of the package in FIG. 1 showing an opening sequence for the package.

FIG. 5 is a side view of a container according to a second exemplary embodiment with two separate container parts.

FIG. 6 is a side view of a container according to a third exemplary embodiment with resilient means.

FIG. 7 is a side view of a container according to a fourth exemplary embodiment with opening device.

FIGS. 8a-8c show isometric views of a container according to a fifth exemplary embodiment o in a closed position (FIG. 8a), an open position (FIG. 8b) and a completely open position (FIG. 8c).

FIGS. 9a and 9b show isometric views of a sixth exemplary embodiment with different shape of the component compartment.

FIGS. 10a and 10b show isometric views of a seventh exemplary embodiment with different shape of the component compartment.

FIGS. 11a and 11b show isometric views of a eighth exemplary embodiment with different shape of the component compartment.

FIGS. 12a and 12b show isometric views of a ninth exemplary embodiment with different shape of the component compartment and without an access channel.

FIGS. 13a and 13b show isometric views of a tenth exemplary embodiment with different shape of the component compartment.

FIGS. 14a and 14b show isometric views of a eleventh exemplary embodiment with different shape of the component compartment.

### DESCRIPTION OF EXEMPLARY EMBODIMENTS OF THE INVENTIVE CONCEPT

Below, a container 1 for the purpose of keeping a dental component sterile from the packaging of the component to the final user is described, which normally is to be encased in an outer container. In the following description the expression ‘package’ will be used for a container containing a component, and this container may in turn be covered by an outer package, but this is normally not a necessity. Furthermore, the expression ‘container’ is used for the structure directly enclosing or surrounding the component. The expression ‘dental component’ is used to comprise different kinds of components with regard to dental implants, and components used during dental surgery, for example bone screws, screws, abutments, washers, healing abutments etc.

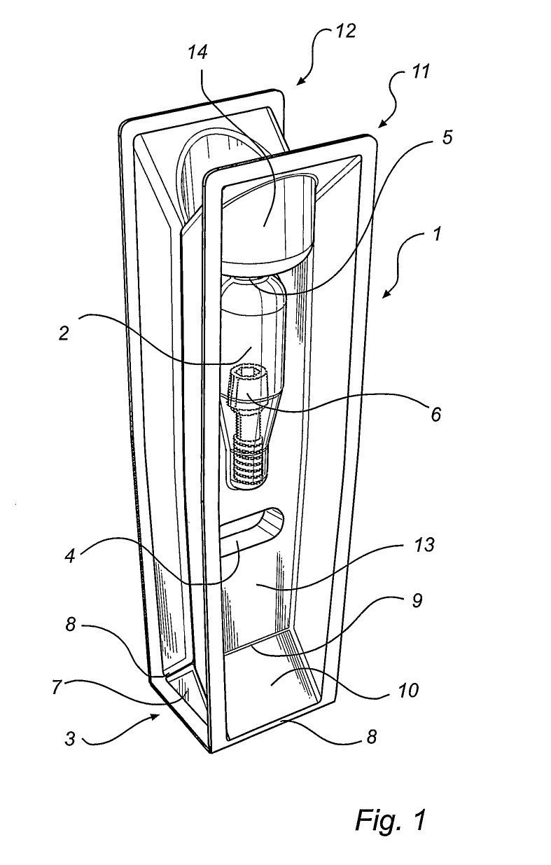

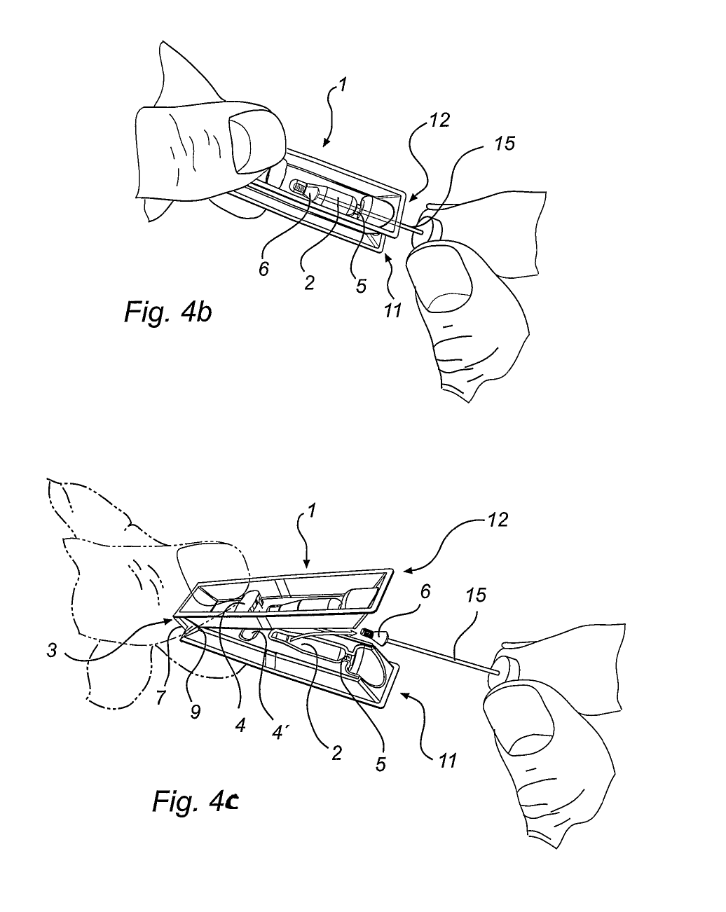

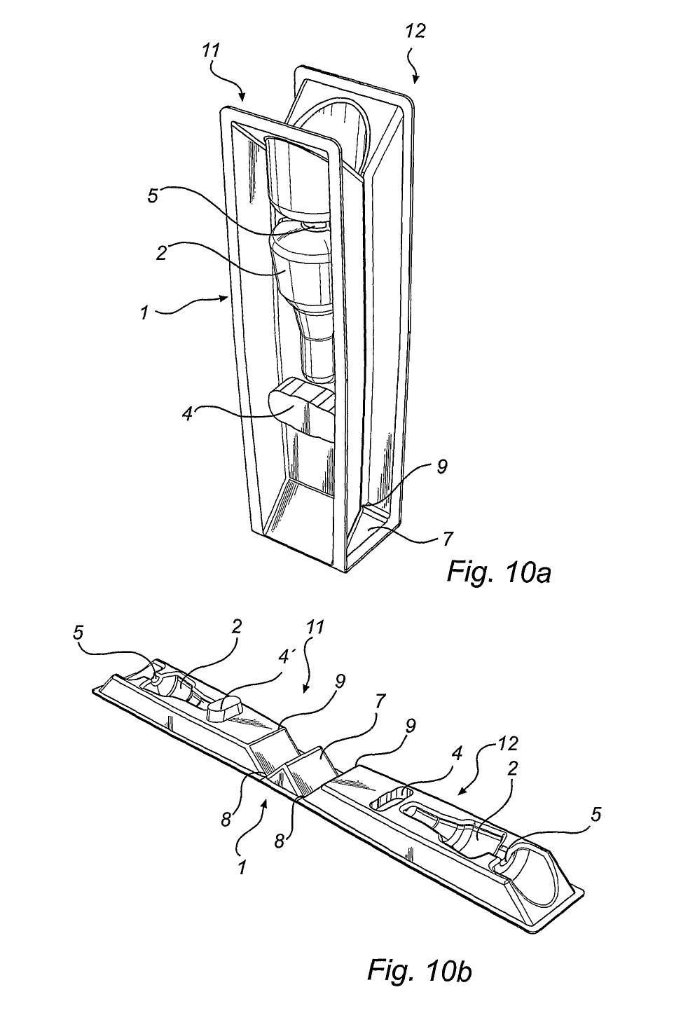

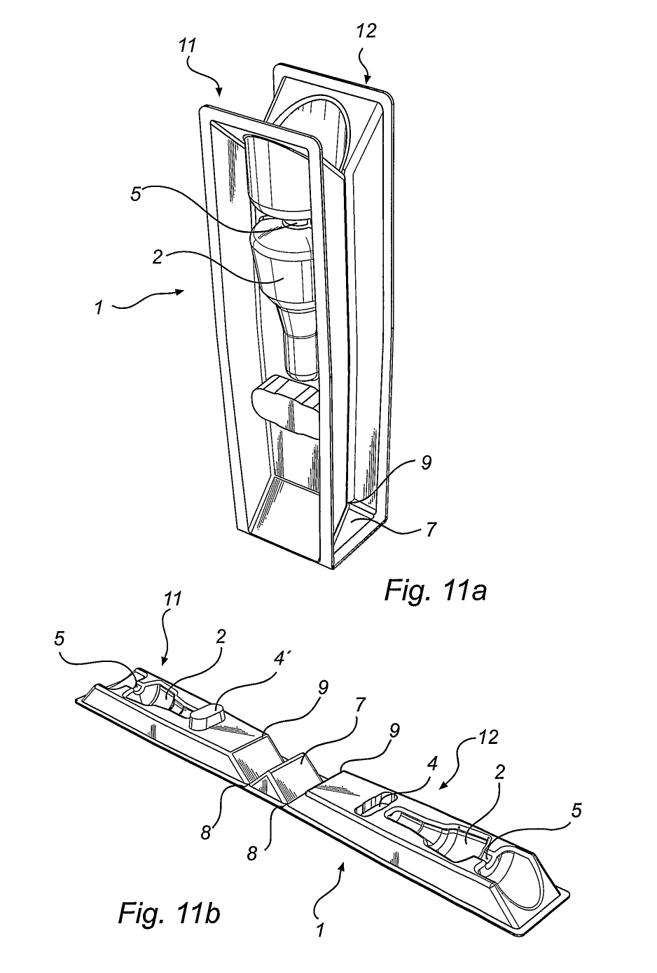

A first embodiment of a container 1 according to the inventive concept will now be described in relation to FIGS. 1 to 4. The inventive concept is embodied by different designs of the package 1, 6 shown in FIGS. 5 to 14.

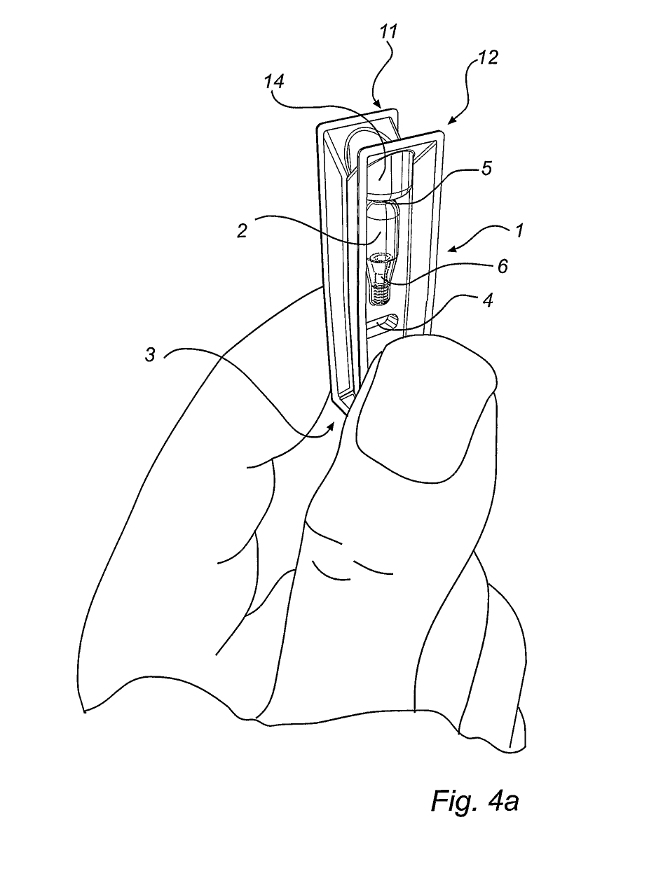

FIG. 1 depicts a first embodiment of a package 1, 6 according to the inventive concept, in which a dental component 6, namely in this case a cover screw, is contained. The package 1, 6 in FIG. 1 consists of a lightweight transparent or at least semi-transparent material, through which the cover screw 6 is readily visible. The purpose of the transparent characteristic is to facilitate the attachment of an external component removal instrument 15 (see FIG. 4b) to the cover screw 6. The instrument 15, particular a mounting tool, is attached to the cover screw 6 by introducing the instrument 15 into the container 1, while the container 1 still is in the closed position. This feature will be discussed in detail below. The instrument 15 may consequently also be structured and adapted for applying the component in the patient, for example screwing an implant into bone tissue, why the instrument also may include a structure for engaging the component rotationally fixed.

The container 1 comprises a first and a second container part 11, 12, which in this embodiment is formed as container halves of corresponding size and shape. In the first embodiment the two halves are connected by a resilient and supporting portion 7 or intermediate connecting part 7. This supporting portion 7 also connects the first and second halves of the container 1 by living hinges 8.

In FIG. 1, the package 1, 6 is shown in a closed position, wherein the first and second container part 11, 12 is attached to each other by a lock 4. In this first embodiment, the locking means 4 are a snap lock formed by a protrusion 4′ in said first part 11, and a corresponding indentation 4″ in said second part 12. In the closed position of the container 1, the first and second parts 11, 12 form a component compartment 2 that contains the dental component 6. It is normally only one component 6 present in the compartment, but in specific embodiments, more than one component may be present in the compartment. In the case with more than one component 6, the components 6 may be extracted simultaneously, if they are connected, or one at a time. The two parts 11, 12 also form an access channel 5 extending from the component compartment 2 to the exterior of the container 1. This access channel 5 provides access, by the above-mentioned external instrument 15, of the component 6 in the container 1 from the outside of the container 1, without the need of opening the container 1. The component compartment 2 is in this embodiment shaped so as to roughly follow the outer shape of the component 6 to be stored therein. The component 6 is accordingly accessible by an external instrument 15 through the access channel 5 already when the container 1 is closed. In order to facilitate said access and to guide the instrument 15 into the engagement part of the component 6, the channel 5 is so designed to conform to the outer contour of the instrument 15, at least partly. To minimize the need for precision handling, the access channel 5 is somewhat wider in the direction facing the mouth 14, and tapering towards the component compartment 2, being of its smallest cross-sectional dimension situated adjacent the compartment 2.

In order to further facilitate the engagement between the instrument 15 and the component 6, the component 6 is preferably turned so within the compartment 2 that its instrument-engaging end is facing the channel 5. It is contemplated that the component 6 even has a bulge or other structure in one or both of the compartment parts 2′, 2″ to hold the component 6 in place. On the other hand, other components 6 may not need such guidance or control, and hence the compartment 2 may be of a more general shape. Since there are several features that may simplify the engagement of the component 6 within the container 1, the need for the container 1 to be made of a transparent material is minimized.

Furthermore, this exemplary embodiment of the container 1, in the closed or folded position thereof, has the outer contour of an elongated rectangular box with a length in the range of 30 to 70 mm, preferably 50 mm and cross section in the range 5×7 mm to 15×20 mm. In the closed position of the package 1, 6, i.e. in the closed position of the container 1 containing the component 6, an interface between the first and second container parts 11, 12 together define a geometrical medial plane. The first and second container parts 11, 12 each present a main portion that is located essentially on a first side and on a second side of the medial plane. In the present embodiment, the container compartment 2 is divided into a first and a second compartment part 2′, 2″ that each is located on the first and on the second side of the medial plane. Furthermore, the access channel 5 is divided into a first and a second channel part 5′, 5″ that each is located on the first and on the second side of the medial plane. The first container part 11 is located on the first side of the medial plane and comprises the first compartment part 2′ and the first channel part 5′. The corresponding situation applies to the second container part 12 etc.

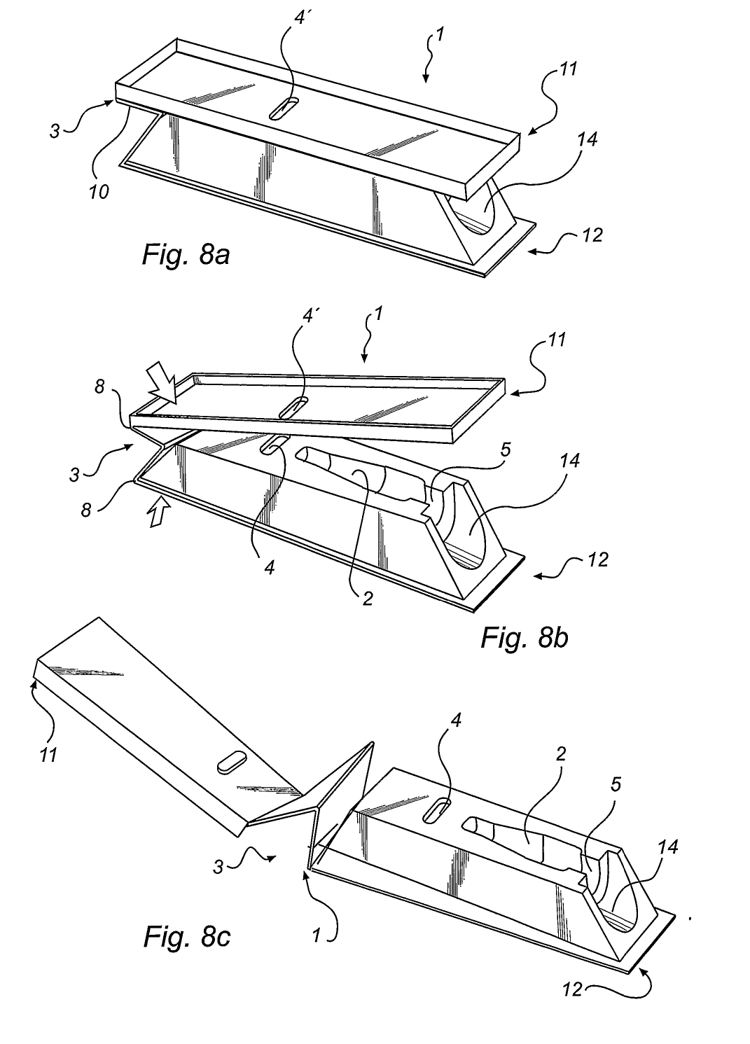

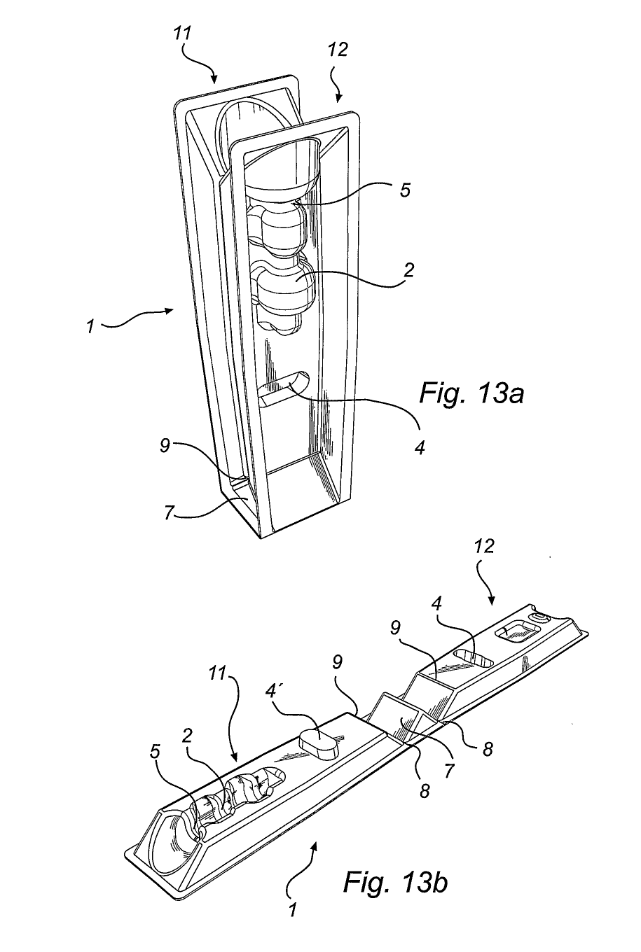

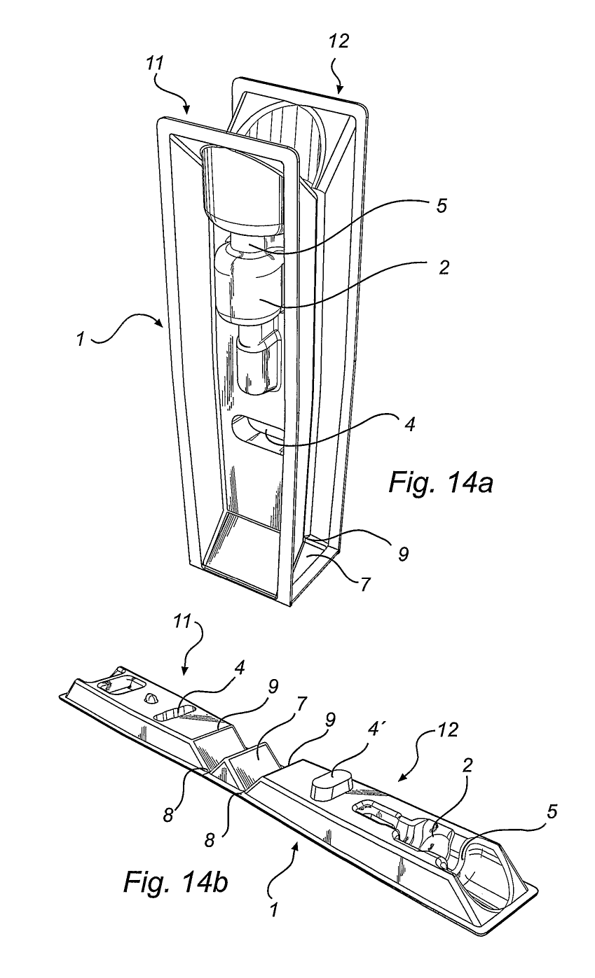

In this embodiment, the package 1, 6 is elongated towards what may be called a mouth or outer channel 14. Each respective channel part 5′, 5″ faces the mouth 14. In turn, each respective compartment part 2′, 2″ is facing the respective channel part 5′, 5″. Each compartment part 2′, 2″ is of equal size and contour. However, in FIGS. 8, 13 and 14 other exemplary embodiments are disclosed, presenting other situations. In FIG. 8, a package 1, 6 is contemplated which has a compartment 2 in only one of the container parts 11, 12. The compartment 2 of FIGS. 13 and 14 (as best seen in FIGS. 13b and 14b) are divided into a first and a second compartment part 2′, 2″. These are however not symmetrically positioned within the container 1, like in FIG. 1. The compartment parts 2′, 2″ are instead positioned asymmetrically around the geometrical medial plane.

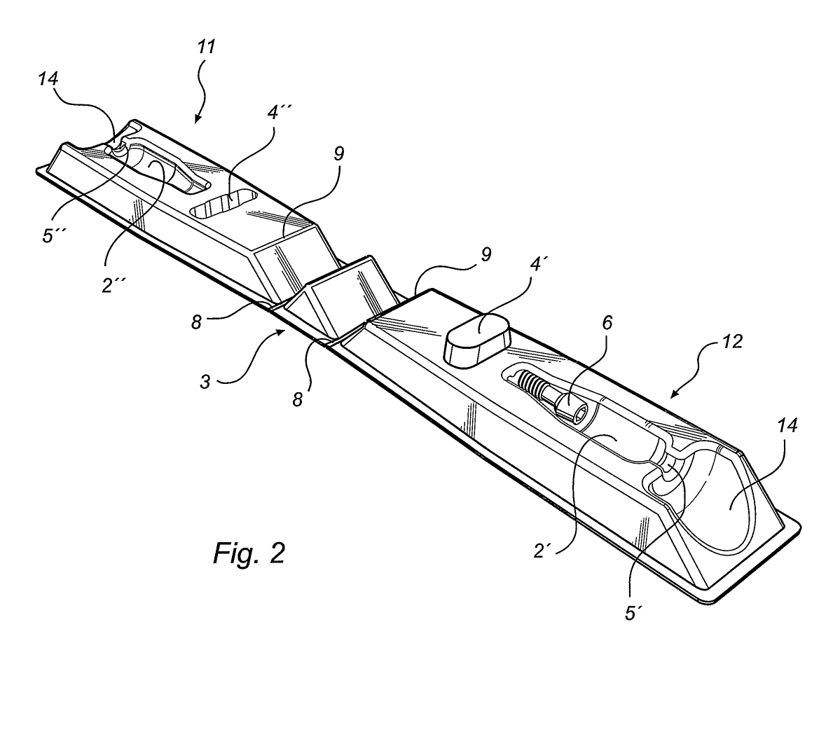

In FIG. 2 the container 1 according to the first embodiment is shown in an initially open, or unfolded, position. Preferably the container 1 is manufactured in this position by thermoforming, vacuum forming or other appropriate method. After the manufacture, the components 6 that are to be stored within the containers 1 may be directly placed therein, possibly after a sterilization step. The container 1 is made in one piece of a plastic material, or other material that is able to facilitate the requirements of manufacturing, cost, sterility, hygiene, etc. This initially open position that is shown in FIG. 2, is not the same open position as when the container is brought to the open position, during use, for extraction of the component 6 from the container 1. Of course, it is possible to open the container back to the initially open position for extraction of the component 6. But it is advantageous to open the container to the position shown in FIG. 4c for extraction of the component from the container 1, since, as will be described below, the container 1 is designed therefore and it gives certain advantages.

FIG. 4a shows the container 1 that is adapted to be held and opened using two fingers of the same hand of the user. In FIG. 4a, the package 1, 6 is picked up by the user. The user has possibly removed an outer container that may have provided further environmental protection. The thumb and the index finger each apply a gripping pressure, but not an opening pressure on the maneuver section 3 of the container 1. The locking force applied by the locking means 4 is thus still larger than the gripping pressure. This will facilitate easy and safe handling of the container 1. The user's other hand may then be used to insert the external instrument 15 through the access channel 5 of the still closed container 1, and engage the component 6. This will limit the risk for contamination or dropping the dental component 6 stored in the container when later opening it. The component is safely engaged by the instrument all through the opening and component extraction process.

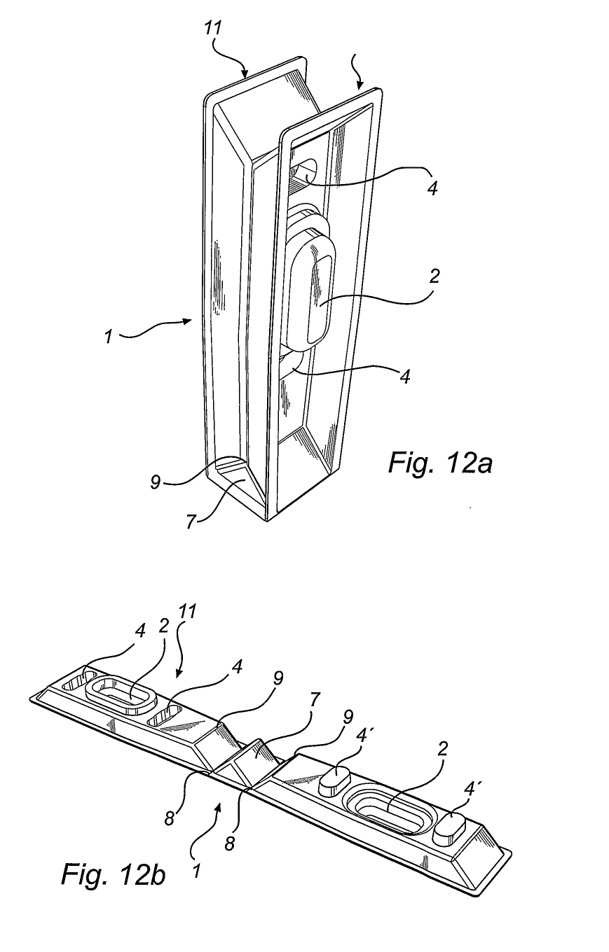

FIG. 4b illustrates the first step in the opening procedure of the package 1, 6, how a container removal instrument 15 is inserted into the package 1 through the access channel 5 for engagement with the instrument engagement structure of the dental component 6, for example an Allen key, i.e. hexagonal, Torx or other suitable structure or other method for attaching the component to the instrument during extraction of the component out from the container. Such other methods could be to use tweezers, magnetic or adhesive material for attaching the component to the instrument. The expression engagement has been used, since, in order to effect the removal by the instrument, there must be some form of component-carrying function present. Consequently, the expression ‘engagement’ is used in a meaning more than just contact between the instrument and the component. Of course it is also possible to use the container for components not having a specific engagement portion, e.g. when a tweezers is used. Alternatively, the container can be used without a channel, which is shown in FIG. 12, e.g. in cases when it is not suitable to use a removal instrument or when the component do not have an engagement structure. In this embodiment the container may be a nontransparent container.

During the above-mentioned component engagement step, shown in FIG. 4b, it may also be advantageous to further fixate the dental component 6 inside the container 1 by pressing, and slightly deforming, the container 1 with the fingers in the area of the component compartment 2. This fixation will facilitate the engagement of the instrument 15 to the component 6. The material from which the container 1 is manufactured is thus preferably somewhat resilient. The container 1 may also be designed to be made thin enough to accomplish the same flexibility.

In FIG. 4c it is illustrated how, in the second step of the opening procedure of the package 1, 6, the maneuver section 3 of the container 1 is compressed to transfer the container 1 into an open position, in which the component 6 may, in a third step, be removed from the container 1 by the external instrument 15.

In side view the resilient means 7 has a triangular shape, this shape will provide a controlled opening of the container 1 regarding position, speed and stability. The resilient means 7 are formed to have material on all surfaces except the rectangular surface facing away from the container 1 in its closed position.

The force applied by the thumb and the index finger is in this step larger than the locking force applied by the locking means 4.

The open position is thus obtained by deforming the resilient means 7, wherein the container parts 11, 12 are rotated relatively to each other and the locking means 4 are released. The deformation is mainly buckling of the triangular base side surfaces. This opening movement is a rotation of the container parts 11, 12 about a pivot axis 9, which in this case also lies in the geometrical medial plane between the parts 11, 12. This pivot axis 9 may preferably be essentially fixed in space, but one may consider embodiments where the position of the pivot axis 9 relative to the container 1 as such actually varies somewhat during the opening procedure, e.g. due to a rolling contact between the two container parts 11, 12. The rolling contact may be compared to rounded ridges that are rolling on one another.

The container I is transferred between the initially open position (FIG. 2) and the closed position (FIG. 1 and FIG. 4a) by folding the container parts 11, 12 at the hinges 8 connecting first and second container parts 11, 12 with the resilient means 7, i.e. rotating the first part 11 around one axis or hinge 8 and rotating the second part around another axis or hinge 8. Thus, in the above-mentioned, exemplary embodiment, the pivot axis 9 does not coincide with the hinges 8. The hinges 8 thus allow the container 1 to be laid flat open initially as is shown in FIG. 2, and then folded to its closed position as is shown in FIG. 4a, where the hinges 8 are located essentially on the outer surface of the closed container 1, and the pivot axis 9 is located in the interior of the container 1.

It should be noted that the component compartment 2 is located between the access channel 5 and the pivot axis 9, and that the locking means 4 are located between the compartment 2 and the pivot axis 9. The locking force, and consequently the pressure needed on the maneuver section 3 for opening the container 1, may be adjusted by moving the position of the locking means 4 in relation to the pivot axis 9. The distance there-between forms a lever, which the longer the lever the more pressure is needed on the maneuver section 3 to open the container 1. Another manner to adjust the pressure needed may be to alter the locking means 4 to either absorb more friction between locking parts 4′, 4″ to have more than one lock 4, or to redesign the locking means 4 to for instance be a snap-in closure. In the present exemplary embodiment, the lock 4 is formed with a male portion 4′ in one of the parts 11, and with a female portion 4″ in the other part 12. The female portion is a generally oval, or round, recess and the male portion is a protrusion with corresponding form and slightly bigger dimensions to receive a press fit between the male and female portion when the male portion is pressed into the female recess. In the case of a snap-in closure, the size of the lip of the snap-in closure determines the pressure to open the container 1.

The maneuver section 3, i.e. the first and second container parts 11, 12, is formed to create a v-shaped recess. The maximum opening angle of the container 1 is in this embodiment directly limited by the angle of the v-shaped recess, which in this embodiment is in the range 20° to 60°, preferably 45°. The above-mentioned resilient means 7 and the maneuver section 3 are formed having complementary shapes.

FIG. 4d shows the container 1, in its open position when the component 6 has been removed there-from.

In this first exemplary embodiment of the inventive concept the maneuver section 3 is provided in one of the short sides of the elongated box shaped container 1. The maneuver section 3 is formed integrated in or in one piece with the container 1.

In the first embodiment of this inventive concept the first and the second container parts 11, 12 of the container 1 are formed with rims at the edges, which makes the package 1 more rigid and stable. The rim may also form the same outer contour for different containers 1 for components within the same size range. Containers 1 having the same outer contour, e.g. rectangular parallelepiped or cylindrical, may all be packaged within the same kind of outer container 1. This may reduce the number of package parts that must be kept in stock by the manufacturer, thus reducing costs.

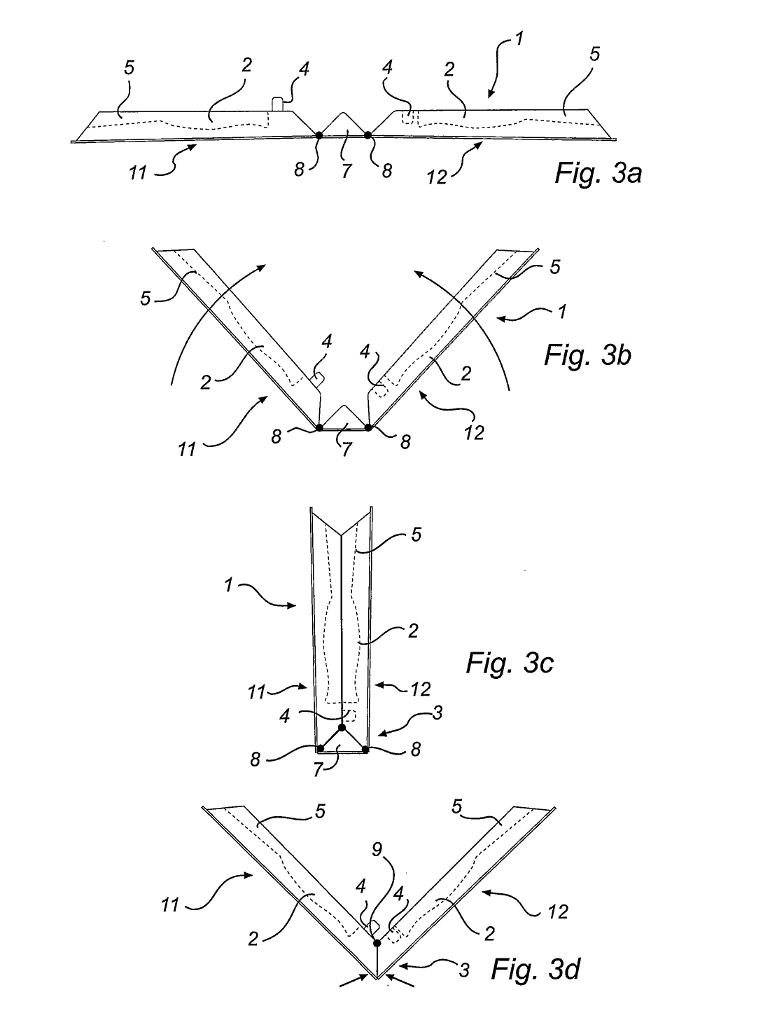

In FIGS. 3a-3d a generalized container 1 is shown. FIG. 3a represents the manufacturing state or an initial open position of the container 1, in which the container 1 lies flat on a surface. A component 6 may in this position of the container 1 be inserted.

FIG. 3b represents the closing maneuver of the container 1, i.e. when the container 1 is transferred from its initial open position to a closed position thereof. It should be noted that the position of the container 1 does not represent a real position in space during this closing maneuver, but is merely a generalized sketch that should be seen in relation to the other figures in the series. The first and the second container parts 11, 12 are pivoted around a respective hinge 8 that surrounds an intermediate part or resilient means 7. The closing maneuver takes place in one two-dimensional geometrical plane.

FIG. 3c represents the closed position of the container 1.

FIG. 3d represents the (component extraction) open position of the container 1. Pressure has been applied to the maneuver section 3, and the resilient means 7 has been compressed, consequently separating the first and the second container parts 11, 12 by pivoting about the pivot axis 9. This opening maneuver takes place in the same two-dimensional geometrical plane as the above-mentioned closing maneuver. Furthermore, the axes or hinges 8 and the pivot axis 9 are parallel to each other. They are also separated from each other.

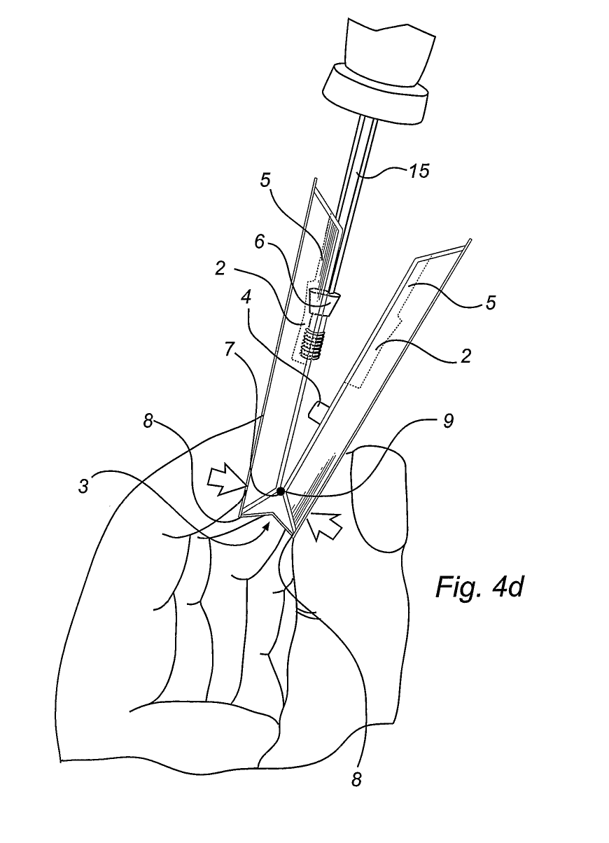

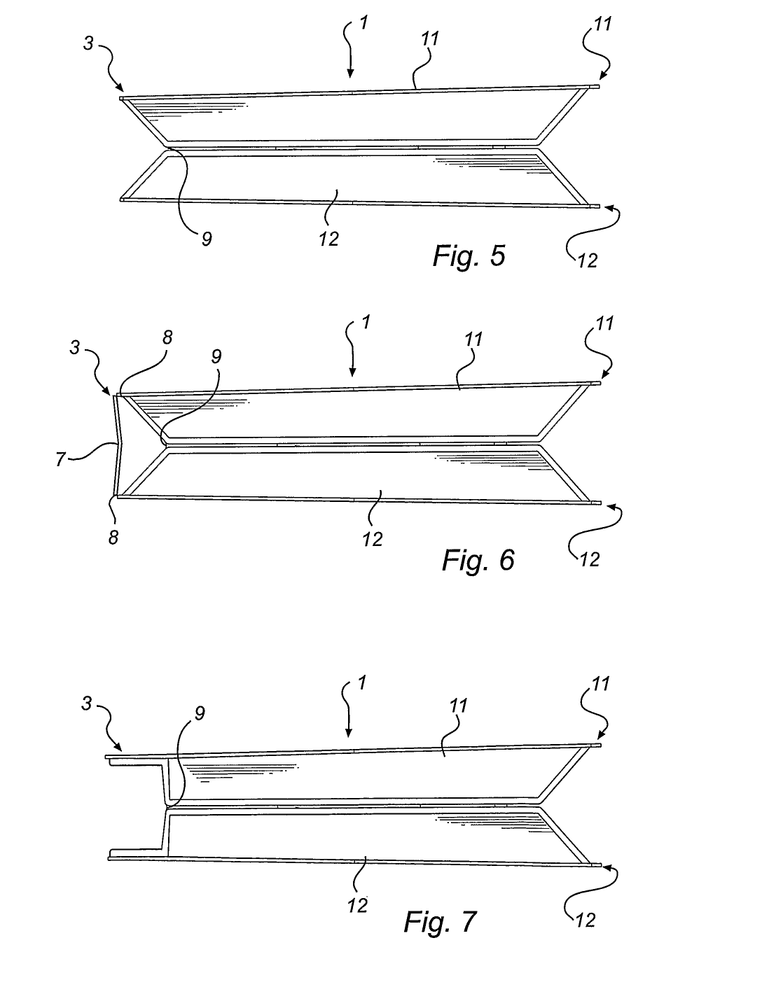

In FIGS. 5-7 other exemplary embodiments of the inventive concept are shown. In FIG. 5, the first and the second container parts 11, 12 are not at all interconnected by resilient means 7. However, the closing and opening maneuvers may still be performed as has been discussed above. In FIG. 6 there are resilient means 7 formed from a plane piece of material connecting the first and the second container parts 11, 12. During opening of the container 1, the resilient means 7 take up some of the opening force in order to stabilize and control the opening maneuver. The plane piece of material is deformed by a combination of buckling and bending during the opening. Also resilient means 7 having other geometrical shape may be used. In FIG. 7, the recess in the maneuver section 3 has a rectangle-shaped design. However, the maneuver section 3 still works as a lever in relation to the pivot axis 9, and also this exemplary embodiment follows the above-described closing and opening maneuvers.

The access channel 5 for attaching the instrument to the component 6 within the compartment 2 may alternatively be formed in only one of the first and the second container parts 11, 12. Such an exemplary embodiment is shown in FIGS. 8a-8c. In this embodiment, the first and second container parts 11, 12, forming the container 1, are designed to form a thin lid 11 and a main body 12. The main body 12 also contains the whole compartment 2 and the whole access channel 5. The lid closes of the upper part of the compartment 2 and the channel 5. In FIG. 8b it is shown how the container is opened by applying a pressure to the maneuver section 3. In FIG. 8c the container 1 according to this embodiment is shown in a completely open position.

When the container 1 is positioned horizontally, the channel 5 may be provided at an angle out from the horizontal plane. One purpose for this may be to achieve easier access to the component 6 when the container for instance lies on a table.

It is possible to provide the maneuver section 3 on other sides of the container 1, e.g. on a long side.

In particular the inventive concept may be carried out as a conventional folding blister package or clamshell package.

Alternative ways to open the container 1 is to use the external instrument as a lever 15. When the instrument 15 is inserted into the container 1, it is possible to pivot the instrument 15 about its tool tip or engagement portion, to open the container 1. Another alternative to open the container 1 is to use both hands, one gripping each one of the first and second container parts 11, 12 and thus applying tension forces on the locking means 4.

The outer part 14 of the access channel 5 may be wider than an inner part of the access channel 5, i.e. a part 15 adjacent the compartment 2, which is designed to correspond to a tool part of the instrument 15. The outer part 14 of the access channel 5 may thus be designed to correspond to a handle part of the instrument 15.

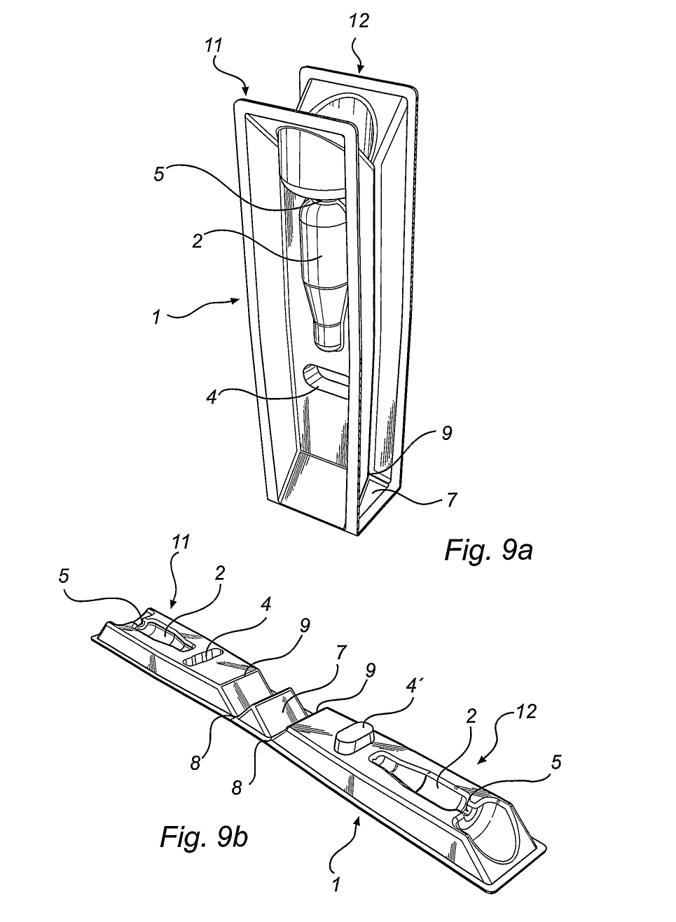

According to one exemplary embodiment of the inventive concept, the component compartment 2 has essentially the same geometrical shape as the component 6 to be stored in the compartment. Three different exemplary embodiments according to this principle are shown in FIGS. 9-11. In another exemplary embodiment of the inventive concept the compartment 2 is designed to be able to receive components 6 having different shapes, i.e. a smaller component stored in this kind of compartment might tumble around within the compartment 2. Such an embodiment is shown in FIG. 12. On the other hand, it is contemplated that the component 6 may be fixed within a compartment 2 of this kind, also when the container 1 is in its open position. The component 6 may have a dimension that for instance is somewhat larger than the corresponding cross-sectional dimension of the compartment 2.

It is furthermore, contemplated that several containers 1 of any one of the above-described exemplary embodiments may be joined, or possibly never separated during manufacture. In such a multi-pack type of container 1 either several components 6 of the same kind may be stored, or different component 6 types that is needed during surgery in a range one coming after the other. The surgeon may in the latter case open the package 1, 6 and have in front of him or her, the components 6 needed for a certain type of procedure.

Combinations of the above-described exemplary embodiments and proposed designs of the container 1 shall be considered to fall within the scope of the present inventive concept.

1. A medical package including a container (1) and a medical component (6), in particular a dental implant component,
wherein said container (1) in a closed position thereof forms a component compartment (2) for said medical component (6) and an access channel (5) for an external instrument, said access channel (5) being dimensioned to prevent said component (6) from passing through the access channel (5) and to allow said instrument to be inserted through the channel (5) and brought into engagement with the component (6) while the container (1) is in its closed position; and
wherein said container (1) is transferable to an open position while maintaining said engagement of the external instrument with the component (6);
whereby said component (6), in the open position of said container (1), is removable from the container (1) by said external instrument.

2. A package as claimed in claim 1, wherein the component compartment (2) is structured and adapted to hold said medical component (6) in an essentially fixed position within the component compartment (2), when the container (1) is in its closed position.

3. A package as claimed in any of the preceding claims, wherein said medical component (6) presents an engagement portion and said instrument presents an engagement portion, which engagements portions are structured and adapted to be brought into engagement with each other.

4. A package as claimed claim 3, wherein the component compartment (2) is structured and adapted to retain said medical component (6) to such an extent that, in the closed position of the container (1), the engagement portion of the medical component (6) is kept in an access position facing the access channel (5) to be engaged by the instrument.

5. A package as claimed in claim 4, wherein said engagement portion of the medical component (6) and said engagement portion of the instrument are structured and adapted to provide a rotationally fixed engagement with respect to an axis essentially parallel to the access channel (5).

6. A package as claimed in any of the preceding claims, wherein said medical component (6) is structured and adapted to be screwed into a prepared site and wherein said engagement is a rotationally fixed engagement allowing said medical component (6), when removed and still engaged by the instrument, to be screwed at least partly into the prepared site by the use of the same instrument.

7. A package as claimed in any of the preceding claims, wherein said container (1) is structured and adapted to be opened by one hand only, preferably by two fingers of one hand.

8. A package as claimed in any of the preceding claims, wherein said container (1) is structured and adapted to be opened by means of application of a pressure against opposite sides of the container (1).

9. A package as claimed in claim 8, wherein said container (1) is structured and adapted to be opened by means of the application of a pressure against opposite sides of a maneuver section of the container (1), said maneuver section of the container (1) being spaced from the component compartment (2) of the container (1), whereby said pressure is not applied on the compartment (2) when the container (1) is opened.

10. A package as claimed in any of the preceding claims, wherein said container (1) comprises first and second container parts being pivotable relatively to each other about a pivot axis for bringing the container (1) to its open position, by the application of a pressure on a maneuver section of the container (1), said maneuver section and said component compartment (2) being located on opposite sides of said pivot axis.

11. A package as claimed in claim 10, wherein said first container part is located essentially entirely on one side of a plane coinciding with said pivot axis and wherein said second container part is located essentially entirely on an opposite side of said plane.

12. A package as claimed claim 11, wherein an interface between said first and second container parts together define a geometrical medial plane of the closed container (1);
said pivot axis is located essentially in said medial plane;
said medial plane and said pivot axis together define a geometrical lateral plane coinciding with the pivot axis and being transverse to said medial plane;
said first and second container parts each present a main portion being located essentially on a first side and a second side, respectively, of said medial plane;
said main portions are located on a first side of said lateral plane facing away from the maneuver section of the container (1),
said main portions form said component compartment (2) and said channel (5) of the container (1);
said first and second container parts each present also a maneuver portion located on an opposite second side of said lateral plane, said maneuver portions together forming said maneuver section of the container (1) and being adapted to be rotated against each other upon application of said pressure.

13. A package as claimed in any one of claims 10-12, wherein the component compartment (2) is located between the access channel (5) and said pivot axis.

14. A package as claimed in any one of claims 10-13, wherein said access channel (5) and said pivot axis are located essentially in a common geometrical plane.

15. A package as claimed in claim 10, wherein said pivot axis is located in a geometrical medial plane formed by the interface of said first and said second portions of the container (1) in the closed position thereof.

16. A package as claimed in any one of the preceding claims, wherein said access channel (5) is tapering towards the component compartment (2).

17. A package according to claim 16, wherein the access channel (5) presents a smallest cross section situated adjacent to the component compartment (2).

18. A package according to any one of the preceding claims, wherein at least a portion of the access channel (5) is designed so as to correspond to an outer contour of the instrument, whereby said channel (5) provides a guide for positioning said instrument in relation to said component (6).

19. A package according to any one of the preceding claims, wherein said container (1) is formed as a one-piece unit.

20. A package according to any one of the preceding claims, wherein said container (1) comprises:
a first part and a second part forming together said component compartment (2), and
an intermediary part connecting said first and second portions.

21. A package according to claim 20, wherein said intermediary portion is hingedly connected to the first and second container part.

22. A package as claimed in claim 20 or 21, wherein said first, second and intermediary parts are formed as a one-piece unit.

23. A package as claimed in any one of claims 20 to 22, wherein said intermediary container part forms a resilient support means for obtaining movement stabilization when the container (1) is brought from its closed position to its open position.

24. A package as claimed in any one of the preceding claims, further comprising locking means for preventing unintentional opening of the container (1).

25. A package as claimed in claim 24, wherein said locking means are formed in one piece with the container (1).

26. A package as claimed in claim 24 or 25, wherein said locking means comprises at least one of frictional locking means and snap lock means.

27. A package as claimed in any one of the preceding claims, wherein the container (1) at least partly is made of a transparent material.

28. A package as claimed in any one of the preceding claims, wherein the container (1) is a blister-type container (1).

29. A package as claimed in any one of the preceding claims, wherein said medical component (6) is a component of a dental implant system.

30. A package as claimed in any one of the preceding claims, wherein no part of said component (6), in the closed position of said container (1), protrudes outside of said container (1).

31. A medical container (1) for containing a medical component (6), in particular a dental implant component, wherein said container (1) in an initial open position thereof is adapted to receive said component (6), said container (1) in a closed position thereof forms a component compartment (2) for said medical component (6), and said container (1), in a component extraction position thereof, is adapted to present said component compartment (2) for component (6) extraction there-from,
wherein said container (1) is transferable from said initial open position to said closed position in one geometrical plane, and is transferable from said closed position to said component extraction position in essentially the same geometrical plane.

32. A medical container (1) as claimed in claim 31, wherein said container (1) in the closed position thereof further forms an access channel (5) for an external instrument (15), said access channel (5) being dimensioned to prevent said component (6) from passing through the access channel (5) and to allow said instrument to be inserted through the access channel (5) and brought into engagement with the component (6) while the container (1) is in its closed position,
wherein said transfer of the container (1) to said open position is performed while maintaining said engagement of the external instrument with the component (6),
whereby said component (6), in the open position of said container (1), is removable from the container (1) by said external instrument.

33. A medical container (1) as claimed in claim 32, wherein said container (1) and said component (6) forms a medical package according to any one of the claims 1-30.

34. A medical container (1) for containing a medical component (6), in particular a dental implant component, wherein said container (1) in an initial open position thereof is adapted to receive said component (6), said container (1) in a closed position thereof forms a component compartment (2) for said medical component (6), and said container (1), in a component extraction position thereof, is adapted to present said component compartment (2) for component extraction there-from,
wherein said container (1) is transferable from said initial open position to said closed position about hinge means, and is transferable from said closed position to said component extraction open position about a pivot axis, wherein the extension of said hinge means and said pivot axis are essentially parallel to and separated from one another.

35. A medical container (1) as claimed in claim 34, wherein said container (1) in the closed position thereof further forms an access channel (5) for an external instrument (15), said access channel (5) being dimensioned to prevent said component (6) from passing through the access channel (5) and to allow said instrument to be inserted through the access channel (5) and brought into engagement with the component (6) while the container (1) is in its closed position,
wherein said transfer of the container (1) to said open position is performed while maintaining said engagement of the external instrument with the component (6),
whereby said component (6), in the open position of said container (1), is removable from the container (1) by said external instrument.

36. A medical container (1) as claimed in claim 35, wherein said container (1) and said component (6) forms a medical package according to any one of the claims 1-30.

37. A medical container (1) for containing a medical component (6), in particular a dental implant component, wherein said container (1) comprises an maneuver section, a first body, and a second body (11, 12), said first and second body (11, 12) being pivotable relatively to each other about a pivot axis (9) between a closed position and an open position in which the component (6) is extractable from the package, wherein in the closed position,
the first and second body (11, 12) form a component compartment (2) for containing said component (6), and wherein the pivot axis (9) is provided between the maneuver section (3) and the component compartment (2), such that an opening force applied to said maneuver section (3) is transferred via the pivot axis (9) for bringing the first and second body (11, 12) from the closed position to the open position.

38. A medical container (1) as claimed in claim 37, wherein said container (1) in the closed position thereof further forms an access channel (5) for an external instrument (15), said access channel (5) being dimensioned to prevent said component (6) from passing through the access channel (5) and to allow said instrument to be inserted through the access channel (5) and brought into engagement with the component (6) while the container (1) is in its closed position,
wherein said transfer of the container (1) to said open position is performed while maintaining said engagement of the external instrument with the component (6),
whereby said component (6), in the open position of said container (1), is removable from the container (1) by said external instrument.

39. A medical container (1) as claimed in claim 38, wherein said container (1) and said component (6) forms a medical package according to any one of the claims 1-30.

40. A medical package system comprising:
a package including a container (1) and a medical component (6), in particular a dental implant component, and
an instrument for removing said component (6) from the container (1),
wherein said container (1) in a closed position thereof forms a component compartment (2) for said medical component (6) and an access channel (5) for said instrument, said access channel (5) being dimensioned to prevent said component (6) from passing through the access channel (5) and to allow said instrument to be inserted through the channel (5) and brought into engagement with the component (6) while the container (1) is in its closed position; and
wherein said container (1) is transferable to an open position while maintaining said engagement of the instrument with the component (6);
whereby said component (6), in the open position of said container (1), is removable from the container (1) by said instrument.

41. A package system as claimed in the preceding claim, wherein said package is a medical package according to any one of the claims 1-30.

42. A package system as claimed in any one of the claims 40-41, wherein said component (6) presents an engagement portion and said instrument presents an engagement portion, which engagement portions are structured and adapted to be brought into engagement with each other.

43. A package system as claimed in the preceding claim, wherein the component compartment (2) is structured and adapted to hold said component (6) in place to such an extent that, in the closed position of the container (1), the engagement portion of the component (6) is kept in an access position facing the access channel (5) to be engaged by the instrument.

44. A package system as claimed in the preceding claim, wherein said engagement portion of the component (6) and said engagement portion of the instrument are structured and adapted to provide a rotationally fixed engagement with respect to an axis essentially parallel to said access channel (5).

45. A package system as claimed in any of the claims 40-44, wherein said medical component (6) is structured and adapted to be screwed into bone tissue and wherein said engagement is a rotationally fixed engagement allowing said component (6), when removed and still engaged by the instrument, to be screwed at least partly into bone tissue by the use of the instrument.

46. A package system according to the preceding claim, wherein at least a portion of the access channel (5) is designed so as to correspond to an outer contour of the instrument, whereby said channel (5) provides a guide for positioning said instrument in relation to said component (6).

47. A method for removing a medical component (6), in particular a dental implant component, from a container (1), comprising:
in a closed position of said container (1), inserting an instrument through an access channel (5) of said container (1) for bringing said instrument into engagement with the component (6);
bringing said container (1) to an open position while maintaining said engagement; and
removing the component (6) from the container (1) by said instrument.

48. A method as claimed in the preceding claim, comprising:
initially holding the closed container (1) with one hand and the instrument with the other hand; and
performing the opening of the container (1) by the use of said one hand only.

49. A method for removing a medical component (6), in particular a dental implant component, from a container (1), said container (1) comprising first and second container parts which form a compartment (2) for said component (6) and which are pivotable relatively to each other about a pivot axis for bringing the container (1) to its open position, comprising:
bringing the container (1) from its closed position to its open position by the application of a pressure on a maneuver section of the container (1), wherein said maneuver section and said component compartment (2) being located on opposite sides of the pivot axis; and
removing the component (6) from the opened container (1).

50. A method as claimed in the preceding claim, further comprising:
in the closed position of the container (1), inserting an instrument through an access channel (5) of said container (1) for bringing said instrument into engagement with the component (6);
bringing said container (1) to its open position by the application of said pressure on the maneuver section while maintaining said engagement; and
removing the component (6) from the container (1) by said instrument.

51. A method as claimed in any one of the claims 47-50, further comprising:
after removal of the medical component (6) from the container (1), especially a dental implant component, inserting said component (6) into the alveolar bone tissue of a patient.
